(19) United States
(12) Patent Application Publication
(10) Pub. No.: US 2025/0321234 A1
(43) Pub. Date: Oct. 16, 2025

(54) METHOD FOR DETECTING ANTI-DRUG ANTIBODIES AGAINST SELF-ASSEMBLING TRIMERIC BIOLOGICS USING AN AFFINITY CAPTURE ELUTION-PROTEIN A/G ASSAY

(71) Applicant: INmune Bio Inc., Boca Raton, FL (US)

(72) Inventors: Jing Liu, Indianapolis, IN (US); Joshua S. Schoonover, Boca Raton, FL (US); Raymond J. Tesi, Boca Raton, FL (US)

(73) Assignee: INmune Bio Inc., Boca Raton, FL (US)

(21) Appl. No.: 19/199,221

(22) Filed: May 5, 2025

Related U.S. Application Data
(60) Provisional application No. 63/634,665, filed on Apr. 16, 2024.

(51) Int. Cl. G01N 33/68

(52) U.S. Cl. CPC G01N 33/6854; G01N 2333/525

(57) ABSTRACT

A method and kit are provided for detecting anti-drug antibodies (ADAs) against self-assembling trimeric biologics, such as XPro1595, a pegylated variant of soluble human Tumor Necrosis Factor, in biological samples, particularly human serum. The method employs an Affinity Capture Elution-Protein A/G (ACE-AG) assay that overcomes non-specific reagent interactions inherent in conventional bridging and standard ACE assays due to the biologic's dynamic monomer exchange. The assay utilizes a two-plate system: a streptavidin-coated plate with biotinylated trimeric biologic captures ADAs, which are eluted, neutralized, and transferred to a protein A/G-coated plate for specific immunoglobulin capture and detection with a sulfo-tagged trimeric biologic via electrochemiluminescence. The kit includes pre-coated plates, labeled biologics, elution and neutralization solutions, assay buffer, confirmatory reagent, and instructions. The assay offers a robust, specific, and sensitive solution for immunogenicity testing of trimeric biologics.

### BACKGROUND

### Field of the Invention

This invention relates to the field of bioanalytical assays for immunogenicity testing, specifically methods and kits for detecting anti-drug antibodies (ADAs) in biological samples. More particularly, the invention pertains to an affinity capture elution assay utilizing protein A/G for the detection of ADAs against self-assembling trimeric biologics, such as pegylated, engineered variants of soluble human Tumor Necrosis Factor, which pose unique challenges due to their dissociative and associative monomeric interactions.

### Background Art

Immunogenicity testing is a critical component of biotherapeutic development, as the presence of anti-drug antibodies (ADAs) can impact the safety and efficacy of biologic drugs. The detection of ADAs is essential for ensuring patient safety and maintaining the therapeutic efficacy of biologics, as ADAs can neutralize the drug's activity or trigger adverse immune reactions. Standard methods for detecting ADAs, such as bridging assays, typically employ biotinylated and labeled (e.g., sulfo-tagged) forms of the drug to capture and detect ADAs in biological samples, such as human serum. These assays rely on the formation of an immune complex between the ADA and the labeled drug reagents, which is then detected through techniques like electrochemiluminescence.

However, certain biologics, particularly those with complex structures such as self-assembling trimers, present significant challenges to conventional ADA detection methods. Self-assembling trimeric biologics, including engineered variants of proteins like soluble human Tumor Necrosis Factor (TNF), e.g., dominant negative (DN)-TNF variants, comprise monomers that can dynamically dissociate and associate. This property causes biotinylated and labeled drug reagents to bind to each other in the absence of ADAs, leading to high background signals and false positives in standard bridging assays. Attempts to mitigate this issue, such as reducing reagent concentrations, often compromise assay sensitivity or drug tolerance, rendering these approaches suboptimal.

Alternative assay formats, such as Affinity Capture Elution (ACE) assays, have been developed to address some limitations of bridging assays. In ACE assays, ADAs are captured by a drug-coated solid support, eluted, and detected on a separate platform. However, for biologics without an Fc region or those prone to aggregation, such as self-assembling trimeric biologics, standard ACE assays may still suffer from non-specific interactions between assay reagents, particularly when capture and detection reagents are not fully separated. Protein A/G, known for its affinity to immunoglobulins (IgGs and IgMs), has been used in some assay designs to enhance specificity, as described in Johnson et al., Scientific Reports, 2021, but these methods typically do not address the unique challenge of monomer exchange in trimeric biologics, leading to persistent background signals. Similarly, methods for detecting antibodies against multi-epitope antigens, such as polyethylene glycol (PEG), have employed elution-based approaches (e.g., Bivi et al., Journal of Immunological Methods, 2020), but lack the two-plate configuration and protein A/G specificity required for trimeric biologics.

Despite these advances, there remains a need for robust, sensitive, and specific ADA detection methods tailored to self-assembling trimeric biologics that avoid non-specific reagent interactions while maintaining high drug tolerance and assay precision.

Conventional methods combining protein A/G or elution-based assays, while effective for certain biologics, do not specifically address the dynamic monomer exchange inherent in self-assembling trimeric biologics, such as XPro1595. For example, existing immunoassays using protein A/G for immunoglobulin capture typically employ single-plate formats or lack the sequential elution and neutralization steps critical for trimeric biologics, resulting in suboptimal specificity and sensitivity. The present invention's novel two-plate ACE-AG assay distinctly separates biotinylated capture and sulfo-tagged detection reagents, with protein A/G ensuring exclusive immunoglobulin capture, thereby eliminating non-specific reagent interactions. This configuration, absent in prior art, provides a tailored solution for trimeric biologics, achieving validated performance metrics, such as sensitivity below 2 ng/mL and drug tolerance up to 200 ng/mL.

### SUMMARY

The disclosure concerns a novel method and kit for detecting anti-drug antibodies (ADAs) against self-assembling multi-meric biologics, such as XPro1595, a pegylated, engineered DN-TNF variant of soluble human Tumor Necrosis Factor (TNF), in biological samples. The method is particularly advantageous for biologics lacking an Fc region, where conventional assays may fail due to non-specific interactions. The method employs an Affinity Capture Elution-Protein A/G (ACE-AG) assay format designed to overcome the challenges posed by the dynamic dissociation and association of monomers in multi-meric biologics, which cause non-specific interactions in conventional bridging and standard ACE assays.

In the examples, a biological sample is contacted with a biotinylated trimeric biologic coated on a first solid support to capture ADAs. The captured ADAs are eluted using an acidic solution and transferred to a second solid support coated with protein A/G, which specifically captures immunoglobulins. Detection is achieved using a sulfo-tagged form of the trimeric biologic, and the resulting signal, preferably measured by electrochemiluminescence, indicates the presence of ADAs. The separation of capture and detection reagents across two solid supports, combined with the use of protein A/G, prevents non-specific binding between biotinylated and sulfo-tagged drug forms, ensuring high specificity.

The method achieves exceptional sensitivity (<2 ng/ml), drug tolerance (up to 200 ng/ml free drug), and robustness, with no hook effect at high ADA concentrations (up to 65,536 ng/ml). It is effective in complex matrices, including hemolyzed and lipemic serum, and maintains stability through multiple freeze/thaw cycles and extended benchtop conditions. The disclosure also encompasses a kit comprising the necessary reagents and instructions for performing the assay, tailored to trimeric biologics like XPro1595. These embodiments offer a reliable, validated approach for immunogenicity testing of biologics with self-assembling properties, addressing a critical need in biotherapeutic development.

### BRIEF DESCRIPTION OF THE DRAWINGS

The accompanying drawings, which are incorporated in and constitute a part of this specification, illustrate embodiments of the invention and, together with the detailed description of the embodiments given below, serve to explain the principles of the invention as claimed.

FIG. 1 is a flowchart depicting the steps of the Affinity Capture Elution-Protein A/G (ACE-AG) assay method for detecting anti-drug antibodies (ADAs) against a self-assembling trimeric biologic.

FIG. 2 is a schematic illustrating the assay apparatus, showing a streptavidin-coated plate with biotinylated trimeric biologic for ADA capture and a protein A/G-coated standard bind plate for specific ADA detection using a sulfo-tagged trimeric biologic.

### DETAILED DESCRIPTION OF EMBODIMENTS

The following detailed description, together with the accompanying drawings, is provided to illustrate exemplary embodiments of the invention and to enable those skilled in the art to make and use the invention. The description sets forth the best mode contemplated for carrying out the invention, which complies with regulatory guidelines for immunogenicity testing, such as the FDA's 2019 Immunogenicity Testing of Therapeutic Protein Products guidance and the EMA's 2017 Guideline on Immunogenicity Assessment of Therapeutic Proteins. The disclosure is not intended to limit the scope, which is defined by the appended claims. Various modifications and equivalents will be apparent to those skilled in the art without departing from the spirit and scope of the invention.

The disclosure provides a method and kit designed to detect anti-drug antibodies (ADAs) against self-assembling multi-meric biologics, such as XPro1595, a pegylated, engineered variant of soluble human Tumor Necrosis Factor (TNF), which forms a trimeric structure, and other dominant negative tumor necrosis factor variant proteins (DN-TNFs), in biological samples, primarily human serum. The invention employs a novel Affinity Capture Elution-Protein A/G (ACE-AG) assay format, which addresses critical challenges in immunogenicity testing for biologics with complex, dynamic structures. The purpose of the invention is to enable accurate, sensitive, and specific detection of ADAs, which are critical for assessing the safety and efficacy of biotherapeutics, particularly those prone to aggregation due to their self-assembling nature. By providing a robust assay and a comprehensive kit, the embodiments facilitate reliable immunogenicity testing in clinical, preclinical, and research settings, ensuring compliance with regulatory standards.

A significant problem addressed by the invention is the non-specific interactions that plague conventional bridging and standard Affinity Capture Elution (ACE) assays when applied to self-assembling multi-meric biologics. These challenges include the dynamic dissociation and association of monomers, which cause non-specific binding between assay reagents in conventional methods. These biologics, exemplified by XPro1595, lack an Fc region and exhibit dynamic monomer exchange, where monomers dissociate and associate, causing biotinylated and sulfo-tagged drug reagents to bind to each other in the absence of ADAs. This leads to high background signals and false positives, rendering standard bridging assays ineffective. Similarly, standard ACE assays, while reducing some non-specificity, struggle with residual reagent interactions, compromising sensitivity and drug tolerance. These limitations hinder accurate ADA detection, which is essential for evaluating the immunogenicity of biotherapeutics. The invention overcomes these challenges by introducing a tailored assay format that prevents non-specific reagent interactions, which contrasts the ACE-AG assay's specificity with the deficiencies of bridging assays.

The solution provided by the invention is a two-plate ACE-AG assay that separates the capture and detection reagents to eliminate non-specific binding, ensuring high sensitivity and specificity. In the method, a biological sample is first contacted with a biotinylated trimeric biologic, such as XPro1595, coated on a streptavidin-coated plate to capture ADAs. The captured ADAs are eluted with an acidic solution, transferred to a second plate coated with protein A/G, which specifically binds immunoglobulins (IgGs and IgMs), and detected using a sulfo-tagged trimeric biologic via electrochemiluminescence. The use of protein A/G enhances specificity by capturing only antibodies, preventing interference from free biologic or reagent complexes. The kit complements the method by providing all necessary components, including pre-coated plates, labeled biologics, elution and neutralization solutions, and instructions, enabling straightforward implementation.

The invention's performance is validated, achieving sensitivity below 2 ng/ml, drug tolerance up to 200 ng/mL, and no hook effect at ADA concentrations up to 65,536 ng/mL. These metrics ensure reliable ADA detection in complex matrices, such as hemolyzed or lipemic serum, and under varied conditions, including multiple freeze/thaw cycles.

### Overview of the ACE-AG Assay

A core principle of the Affinity Capture Elution-Protein A/G (ACE-AG) assay, central to the present invention, is the strategic separation of capture and detection reagents across two distinct solid supports to eliminate non-specific binding. This separation prevents direct interaction between the biotinylated and sulfo-tagged forms of the biologic, which otherwise bind to each other due to the biologic's self-assembling nature, leading to false positives, a critical issue in immunogenicity testing of self-assembling multi-meric biologics like XPro1595. In conventional assays, the dynamic monomer exchange in these biologics causes biotinylated and sulfo-tagged drug reagents to bind each other in the absence of anti-drug antibodies (ADAs), leading to false positives and high background signals. The ACE-AG assay addresses this by employing a biotinylated multi-meric biologic, such as XPro1595, coated on a streptavidin-coated plate as the capture reagent to initially bind ADAs from a biological sample, typically human serum. Subsequently, the detection reagent, a sulfo-tagged multi-meric biologic, is applied on a separate protein A/G-coated plate, which specifically captures immunoglobulins (IgGs and IgMs) after elution. This two-plate system ensures that the biotinylated and sulfo-tagged reagents do not interact directly, preventing non-specific binding and enhancing assay specificity and sensitivity.

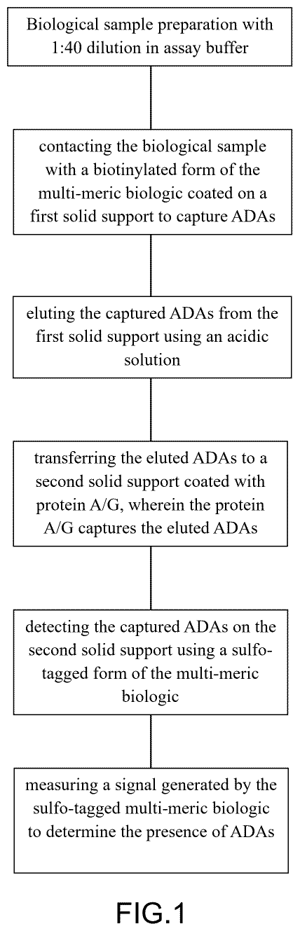

The general process of the ACE-AG assay, outlined in FIG. 1 as a flowchart, involves a series of well-defined steps to detect ADAs with high precision. The process begins with contacting a biological sample, diluted 1:40 in assay buffer to minimize matrix effects, with the streptavidin-coated plate pre-coated with biotinylated trimeric biologic. This step captures ADAs during an overnight incubation at 2-8° C. The captured ADAs are then eluted using 100 μL of 300 mM acetic acid, disrupting the ADA-biologic complex. The acidic eluate (37 μL) is transferred to a protein A/G-coated standard bind plate and neutralized with 37 μL of 0.35M Tris at pH 9.5 in phosphate-buffered saline (PBS) to ensure compatibility with protein A/G binding. The protein A/G captures the immunoglobulins in the neutralized eluate, followed by washing to remove non-specific components. Detection is achieved by adding the sulfo-tagged trimeric biologic, which binds to the captured ADAs, and the resulting electrochemiluminescence signal is measured, preferably using an MSD QuickPlex SQ 120 or similar detector, to quantify ADA presence.

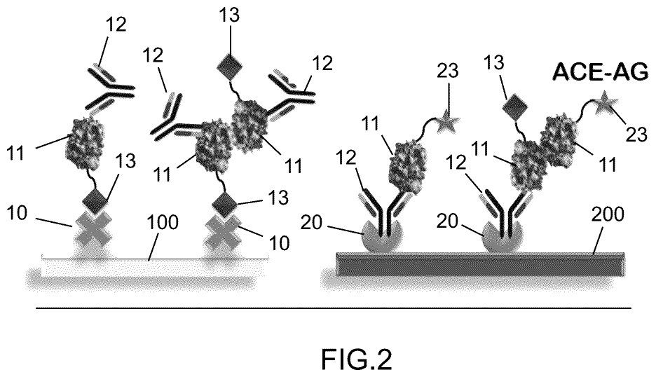

The kit embodiment of the invention complements the method by providing a comprehensive set of reagents and instructions to facilitate assay implementation, as shown in FIG. 2. The kit includes a streptavidin-coated plate pre-coated with biotinylated trimeric biologic for ADA capture, a protein A/G-coated standard bind plate for immunoglobulin capture, a sulfo-tagged trimeric biologic for detection, 300 mM acetic acid for elution, 0.35M Tris pH 9.5 for neutralization, assay buffer for sample dilution, a confirmatory reagent (unlabeled trimeric biologic) for specificity testing, and detailed instructions. These components are designed for stability (e.g., 2-8° C. for reagents, room temperature for plates) and ease of use, ensuring consistent performance in laboratory settings. The instructions guide practitioners through the assay process, from sample preparation to signal measurement, aligning with the workflow in FIG. 1.

By separating the capture and detection phases, as visualized in FIG. 2, the ACE-AG assay prevents the non-specific interactions that compromise conventional assays, achieving validated performance metrics such as sensitivity below 2 ng/ml and drug tolerance up to 200 ng/mL. The flowchart in FIG. 1 provides a clear visual representation of the assay's sequential steps, while the schematic in FIG. 2 illustrates the apparatus configuration, highlighting the roles of the streptavidin and protein A/G plates. Together, these figures elucidate the assay's core principle and process, ensuring that the method and kit can be readily implemented by those skilled in the art for accurate ADA detection in self-assembling trimeric biologics.

With specific reference to FIG. 2, the ACE-AG assay is illustrated as two separate plates, a first plate (100) is coated with streptavidin (10) for capturing a biotinylated protein subunit, such a biotinylated-XPro1595 monomer, having a protein subunit (11) coupled to biotin (13). As shown, there are two practical scenarios where protein (and corresponding antibody) binds the plate, a first including the biotin (13) of a biotinylated-XPro1595 monomer binds streptavidin as depicted in the left side of the plate (100), and a second including protein-protein multimerization (e.g. trimerization with XPro1595) as shown on the right side of the plate (100); in each scenario antibody (12) binds the protein subunits. These antibodies are subsequently washed, as detailed herein, and captured on a second plate (200). Second plate (200) is coated with protein A/G (20), which captures only anti-drug antibodies (not biotinylated protein subunits as these are washed). However, when a sulfo-tag (23) conjugated protein subunit is introduced, it binds the protein A/G-captured ADAs. In the event there was an impurity, such as a biotinylated protein subunit not completely washed from the system, it could bind the anti-drug antibody (ADA), but a multimer complex would be formed with the sulfo-tag (23) conjugated protein subunit reading positive for antibody detection. Overall, the two-plate assay significantly reduces error signals and provides a measurable signal for ADA quantification.

### Detailed Method Steps

The Affinity Capture Elution-Protein A/G (ACE-AG) assay, central to the present invention, involves a meticulously designed sequence of steps to detect anti-drug antibodies (ADAs) against self-assembling trimeric biologics, such as XPro1595 and other DN-TNFs, in biological samples, with human serum being the preferred matrix. The sample preparation step is critical to ensure assay reliability and minimize matrix effects. The biological sample is diluted, e.g. at a ratio of 1:40, in an assay buffer, a dilution factor optimized to balance sensitivity and specificity while reducing non-specific interactions. This diluted sample is used for screening assays to detect ADAs. For confirmatory assays, which assess the specificity of detected ADAs, a parallel sample is pre-incubated with the trimeric biologic, such as XPro1595, to compete with ADA binding. This optional pre-incubation step allows differentiation between specific ADAs and non-specific binding events, enhancing the assay's diagnostic accuracy. The sample preparation process is straightforward, requiring standard laboratory pipetting and mixing techniques, and sets the stage for subsequent assay steps.

Following sample preparation, the capture on the first solid support is initiated. The diluted sample is added to a streptavidin-coated plate, such as a Pierce BSA-blocked Streptavidin plate, which is pre-coated with a biotinylated trimeric biologic, e.g. XPro1595. The biotinylated biologic binds specifically to the streptavidin surface, creating a stable platform for ADA capture. The plate is incubated overnight at 2-8° C. to maximize ADA binding efficiency, allowing ADAs in the sample to form complexes with the biotinylated biologic. After incubation, the plate is washed with a buffer to remove unbound sample components, ensuring that only specifically bound ADAs remain. This step is critical to the assay's specificity, as it leverages the high-affinity biotin-streptavidin interaction to anchor the capture reagent, preventing loss during washing.

The elution step follows capture and involves releasing the bound ADAs from the first solid support. A 100 μL volume of 300 mM acetic acid is applied to the streptavidin-coated plate, disrupting the ADA-biologic complex through acidification. This acidic solution effectively dissociates the ADAs, transferring them into the eluate while leaving the biotinylated biologic bound to the plate. The elution process is optimized to ensure complete ADA release without compromising their integrity, a key factor in maintaining assay sensitivity. The use of 300 mM acetic acid is preferred due to its compatibility with downstream steps.

The transfer and neutralization step involves moving 37 μL of the acidic eluate to a second solid support, a standard bind plate coated with protein A/G and blocked to minimize non-specific binding. To ensure compatibility with protein A/G's immunoglobulin-binding properties, the eluate is neutralized by mixing with 37 μL of 0.35M Tris at pH 9.5 in phosphate-buffered saline (PBS), resulting in a 1:2 dilution. This neutralization step adjusts the pH to a range optimal for protein A/G activity, preserving ADA functionality. The protein A/G-coated plate is designed to capture immunoglobulins specifically, making it a critical component for eliminating interference from non-antibody components in the eluate. The transfer and neutralization process is precise, requiring accurate pipetting to maintain assay reproducibility.

Capture on the second solid support involves the protein A/G-coated plate binding the immunoglobulins (IgGs and IgMs) present in the neutralized eluate. Protein A/G's high affinity for antibodies ensures that only ADAs are retained, enhancing the assay's specificity by excluding free trimeric biologic or other non-specific proteins. The plate is incubated to allow stable binding, typically at room temperature or 2-8° C., followed by washing to remove unbound components. This step is pivotal to the assay's ability to prevent non-specific interactions, a significant improvement over bridging assays. The protein A/G capture ensures that the subsequent detection step targets only ADAs, contributing to the assay's validated performance.

Detection is achieved by adding a sulfo-tagged trimeric biologic, e.g. XPro1595, to the protein A/G-coated plate. The sulfo-tag, a ruthenium-based label, binds specifically to the captured ADAs during incubation, forming a detectable complex. After washing to remove unbound sulfo-tagged biologic, the plate is prepared for signal measurement. This step leverages the specificity of the protein A/G capture to ensure that only ADA-bound sulfo-tagged biologic contributes to the signal, minimizing background noise. The separation of the sulfo-tagged detection reagent from the biotinylated capture reagent across two plates is a key feature that distinguishes the ACE-AG assay from conventional assays.

Signal measurement involves quantifying the electrochemiluminescence signal generated by the sulfo-tagged trimeric biologic, preferably using an MSD QuickPlex SQ 120 or similar instrument. The signal intensity correlates directly with ADA concentration, enabling both qualitative and quantitative assessment of ADA presence in the sample. This final step completes the assay workflow, providing a reliable readout that reflects the assay's high sensitivity and specificity. The electrochemiluminescence method is preferred for its robustness and precision, though alternative detection methods may be adapted. The assay's advantage over bridging assays is avoidance of non-specific reagent interactions obscure signal accuracy, highlighting the ACE-AG assay's ability to deliver clear, specific results for immunogenicity testing of self-assembling trimeric biologics.

### Assay Performance Characeristics

The Affinity Capture Elution-Protein A/G (ACE-AG) assay, as embodied herein, has been rigorously validated to ensure its reliability for detecting anti-drug antibodies (ADAs) against self-assembling trimeric biologics, such as XPro1595, in human serum. The assay's performance metrics, summarized here, demonstrate its exceptional sensitivity, drug tolerance, selectivity, robustness, and stability, making it a robust tool for immunogenicity testing in clinical and preclinical settings.

The assay achieves a sensitivity of less than 2 ng/ml in screening assays and 1.752 ng/ml in confirmatory assays. This high sensitivity enables the detection of low-level ADAs, which is critical for identifying early immune responses to biotherapeutics. Additionally, the assay demonstrates robust drug tolerance, detecting positive controls at 300, 100, and 8 ng/ml in the presence of up to 200 ng/mL of free trimeric biologic. This tolerance ensures reliable ADA detection even in samples with high drug concentrations, a common scenario in therapeutic monitoring. The assay's ability to maintain performance under these conditions underscores its practical utility in real-world applications.

Further validating its performance, the assay exhibits no hook effect at ADA concentrations up to 65536 ng/ml. The hook effect, where high analyte concentrations paradoxically reduce signal intensity, is a common limitation in immunoassays. This characteristic enhances the assay's reliability for samples with varying antibody titers. The assay also demonstrates excellent selectivity with no false positives observed in 10 random lots of human serum from individual donors. When these lots were spiked with 8 ng/ml of surrogate antibody, all samples tested positive in both screening and confirmatory tiers, confirming the assay's ability to specifically detect ADAs without interference from matrix components.

The robustness of the assay is evidenced by its performance in challenging sample matrices, such as hemolyzed and lipemic human serum. These complex matrices, which can interfere with assay performance, did not compromise the ACE-AG assay's ability to accurately detect ADAs, making it suitable for diverse clinical samples. Stability studies further confirm the assay's reliability, with samples remaining stable for ADA detection after 21.2 hours at benchtop conditions and through 8 freeze/thaw cycles. This stability ensures that samples can be handled and stored under standard laboratory conditions without loss of assay performance, enhancing its practicality for routine use.

Precision, a critical aspect of the assay's performance, is demonstrated through consistent inter-run and intra-run results. Validation studies showed good reproducibility across multiple assay runs and within individual runs, ensuring that the assay delivers reliable and repeatable results. This precision, combined with the validated metrics, supports the assay's use in regulatory-compliant immunogenicity testing.

The validated performance metrics of the ACE-AG assay align with regulatory standards for immunogenicity testing, ensuring its suitability for clinical and preclinical applications.

### Kit Components

In another aspect, a comprehensive kit designed to facilitate the Affinity Capture Elution-Protein A/G (ACE-AG) assay for detecting anti-drug antibodies (ADAs) against self-assembling trimeric biologics, such as XPro1595, in biological samples like human serum, is provided. The kit is a complementary embodiment to the assay method, providing all necessary reagents and materials to ensure reliable and reproducible performance in laboratory settings. The components are carefully selected and optimized to support the assay's two-plate format, which separates capture and detection reagents to prevent non-specific binding, a critical feature for trimeric biologics with dynamic monomer exchange.

The kit includes the following components: a streptavidin-coated plate pre-coated with biotinylated trimeric biologic (e.g., XPro1595) for initial ADA capture; a standard bind plate coated with protein A/G and blocked to minimize non-specific binding for specific immunoglobulin capture; a sulfo-tagged trimeric biologic, typically ruthenium-labeled, for detection via electrochemiluminescence; a 300 mM acetic acid solution for eluting ADAs from the first plate; a 0.35M Tris solution at pH 9.5 in phosphate-buffered saline (PBS) for neutralizing the eluate; an assay buffer for diluting samples at a 1:40 ratio to reduce matrix effects; a confirmatory reagent, consisting of unlabeled trimeric biologic, for specificity testing in confirmatory assays; and detailed instructions for performing the assay.

Component stability is a key consideration to ensure the kit's practicality and reliability. The liquid reagents, including the sulfo-tagged trimeric biologic, 300 mM acetic acid, 0.35M Tris pH 9.5, assay buffer, and confirmatory reagent, are stable when stored at 2-8° C., a standard refrigeration temperature for biological reagents. This storage condition preserves the integrity and functionality of these components, allowing for extended shelf life under typical laboratory conditions. The streptavidin-coated and protein A/G-coated plates, pre-coated with their respective reagents, are stable at room temperature, facilitating ease of storage and handling without the need for specialized cold storage. The instructions, provided as a printed manual or digital document, are designed to remain accessible and intact under standard conditions.

The kit is designed for ease of use, enabling both experienced and novice practitioners to perform the ACE-AG assay with minimal training. The pre-coated plates eliminate the need for users to prepare capture and detection surfaces, reducing variability and preparation time. The pre-formulated solutions, such as the acetic acid and Tris buffers, are provided at optimal concentrations (300 mM and 0.35M, respectively), removing the need for on-site dilution or pH adjustment. The assay buffer and confirmatory reagent are ready-to-use, streamlining sample preparation and specificity testing. The instructions provide clear, step-by-step guidance, including incubation times, washing protocols, and signal measurement procedures, ensuring consistency across users.

By including all essential reagents and materials, the kit ensures that the ACE-AG assay can be performed efficiently and reproducibly. The stability of the components supports practical laboratory use, while the pre-configured nature of the plates and solutions minimizes user error, making the kit suitable for high-throughput immunogenicity testing. The inclusion of the confirmatory reagent allows for optional specificity testing, aligning with regulatory requirements. Overall, the kit's design complements the assay method by providing a robust, user-friendly solution for ADA detection in self-assembling trimeric biologics, addressing a critical need in biotherapeutic development.

### Embodiments and Variations

The Affinity Capture Elution-Protein A/G (ACE-AG) assay is designed with flexibility to accommodate various multi-meric biologics, sample types, and assay configurations, ensuring broad applicability in immunogenicity testing. In particular, with the illustrated example being XPro1595 (pegipanermin), the ACE-AG assay is particularly suitable for trimeric biologics. Regarding biologic specificity, in an illustrated embodiment the assay is optimized for XPro1595, a pegylated, engineered variant of soluble human Tumor Necrosis Factor (TNF), a dominant negative TNF (DN-TNF) variant. XPro1595's self-assembling trimeric structure, characterized by dynamic monomer exchange, poses unique challenges due to non-specific interactions between biotinylated and sulfo-tagged reagents in conventional assays. However, the assay's design applies to other self-assembling trimeric biologics, such as members of the TNF superfamily or other proteins with three monomeric subunits capable of reversible dissociation and association. For example, adapting the assay for a different trimeric biologic, such as a TNF-related apoptosis-inducing ligand (TRAIL), involves preparing biotinylated and sulfo-tagged forms of the biologic and adjusting incubation times or reagent concentrations based on its monomer exchange kinetics. This flexibility, supported by the kit's customizable reagents, ensures the assay's utility across diverse trimeric biologics.

For sample types, the assay has been rigorously validated for use with human serum due to its prevalence in clinical immunogenicity studies. The validation process, detailed in the assay's performance metrics, confirmed reliable ADA detection in human serum, including complex matrices like hemolyzed and lipemic serum, ensuring robustness in real-world applications. The sample preparation step, involving a 1:40 dilution in assay buffer, is optimized for serum to minimize matrix effects while maintaining sensitivity.

While the assay is validated for human serum, its design supports adaptation for other biological matrices, such as plasma or cerebrospinal fluid, with minimal optimization. For plasma, a 1:20 dilution in assay buffer may be used to account for higher viscosity, followed by extended washing (e.g., five washes) to reduce matrix interference. For cerebrospinal fluid, a 1:10 dilution and reduced incubation time (e.g., 4 hours at 2-8° C.) on the first plate can enhance sensitivity due to lower protein content. These adjustments, within the skill of the art, leverage the kit's assay buffer and washing protocols to maintain performance metrics, such as sensitivity below 2 ng/mL, as validated for serum. The assay's robustness in hemolyzed and lipemic serum further supports its adaptability, ensuring reliable ADA detection across diverse matrices.

The choice of solid supports further enhances the assay's versatility. The method employs a streptavidin-coated plate for initial ADA capture with biotinylated trimeric biologic and a standard bind plate coated with protein A/G for immunoglobulin capture. These plates are preferred for their high binding affinity and low non-specific interactions, but the assay allows substitution with equivalent alternatives, such as avidin-coated plates for the first support or high-bind plates for the second. Avidin, like streptavidin, supports robust biotin binding, while high-bind plates can effectively anchor protein A/G, maintaining the assay's specificity. This flexibility, supported by the kit's modular design, ensures that practitioners can select solid supports based on availability or laboratory preferences without compromising performance.

In terms of solutions, the assay specifies 300 mM acetic acid for elution and 0.35M Tris at pH 9.5 in phosphate-buffered saline (PBS) for neutralization due to their compatibility with ADA stability and protein A/G binding. However, alternative elution solutions, such as glycine-HCl, or neutralization solutions, such as NaOH, may be used provided they maintain ADA integrity and support protein A/G functionality. These alternatives allow customization based on laboratory resources or specific biologic requirements, with the kit's pre-formulated solutions serving as the default for consistency. The flexibility in solution choice ensures that the assay can be adapted to various experimental conditions while preserving the core principle of specific ADA detection.

The assay's detection methods are similarly adaptable, with electrochemiluminescence, preferably using an MSD QuickPlex SQ 120, as the primary approach due to its sensitivity and precision. This method leverages the sulfo-tagged trimeric biologic's ruthenium label for robust signal generation. However, alternative detection methods, such as fluorescence or chemiluminescence, can be employed by substituting the sulfo-tagged biologic with appropriately labeled reagents, provided the detection system maintains comparable sensitivity. This adaptability, supported by the kit's modular reagent design, allows laboratories to use existing equipment, broadening the assay's accessibility while maintaining performance, as visualized in the assay workflow.

The assay's primary detection method, electrochemiluminescence using an MSD QuickPlex SQ 120, provides high sensitivity and precision. Alternative detection methods, such as fluorescence or chemiluminescence, are supported by substituting the sulfo-tagged trimeric biologic with a fluorescein- or horseradish peroxidase-labeled biologic, respectively. For fluorescence, a plate reader with excitation/emission at 494/520 nm can detect fluorescein-labeled ADAs, requiring calibration to achieve a signal-to-noise ratio comparable to electrochemiluminescence. For chemiluminescence, a luminol-based substrate and a luminometer can be used, with extended washing to minimize background. These alternatives, compatible with the kit's modular design, allow laboratories to utilize existing equipment while maintaining the assay's performance.

The inclusion of confirmatory assays is optional and can be tailored to specific study needs. Confirmatory assays involve pre-incubating the sample with the trimeric biologic to assess ADA specificity, a step included in the kit's confirmatory reagent. This option is particularly valuable for regulatory-compliant studies requiring differentiation between specific and non-specific binding. Laboratories can choose to perform only screening assays or include confirmatory assays based on study objectives, with the kit providing the necessary reagent for flexibility. The modular design of the assay and kit supports this customization, ensuring that the ACE-AG assay can be implemented across diverse trimeric biologics, sample types, and experimental configurations, enhancing its utility in immunogenicity testing.

### Example Implementation

The Affinity Capture Elution-Protein A/G (ACE-AG) assay, as embodied in the present invention, is executed using a comprehensive kit that simplifies the detection of anti-drug antibodies (ADAs) against self-assembling trimeric biologics, such as XPro1595, in human serum. The kit, containing pre-coated plates, reagents, and detailed instructions, enables practitioners to perform the assay with precision and consistency. The following step-by-step example outlines the procedure for performing both screening and confirmatory assays, leveraging the kit's components to achieve reliable ADA detection. Each step aligns with the assay's validated workflow.

The assay begins with sample preparation, where a human serum sample is diluted at a 1:40 ratio in the assay buffer provided in the kit. This dilution, critical for minimizing matrix effects, is performed by mixing 10 μL of serum with 390 μL of buffer, ensuring a total volume suitable for assay processing. For the screening assay, the diluted sample is used directly. For the confirmatory assay, a parallel aliquot of the diluted sample is pre-incubated with the kit's confirmatory reagent, an unlabeled trimeric biologic (e.g., XPro1595), at a concentration sufficient to compete with ADA binding, typically 200 ng/mL. This pre-incubation step assesses the specificity of detected ADAs by comparing signals with and without the confirmatory reagent. The diluted samples (screening and confirmatory) are then added to the kit's streptavidin-coated plate, pre-coated with biotinylated XPro1595, using a multichannel pipette to dispense 100 μL per well, as outlined in the assay workflow.

The first plate is incubated overnight at 2-8° C. to allow ADAs in the sample to bind to the biotinylated trimeric biologic, maximizing capture efficiency. After incubation, the plate is washed three times with a wash buffer (e.g., PBS with 0.05% Tween-20) to remove unbound components, ensuring only specifically bound ADAs remain. This washing step is critical to maintaining assay specificity. Next, 100 μL of the kit's 300 mM acetic acid solution is added to each well to elute the captured ADAs, disrupting the ADA-biologic complex. The plate is incubated briefly (e.g., 5-10 minutes) at room temperature to ensure complete elution, and the eluate is collected. This elution step transfers ADAs into a solution ready for the next phase of the assay.

Using a pipette, 37 μL of the acidic eluate is transferred to the kit's second solid support, a standard bind plate pre-coated with protein A/G and blocked to minimize non-specific binding. Immediately, 37 μL of the kit's 0.35M Tris solution at pH 9.5 in phosphate-buffered saline (PBS) is added to each well to neutralize the eluate, resulting in a 1:2 dilution and a pH suitable for protein A/G binding. This neutralization step ensures that ADAs remain functional for capture by protein A/G, which specifically binds immunoglobulins (IgGs and IgMs). The plate is incubated for 1-2 hours at room temperature or 2-8° C. to allow stable binding, followed by three washes to remove unbound components, maintaining the assay's high specificity.

For detection, 100 μL of the kit's sulfo-tagged trimeric biologic (e.g., ruthenium-labeled XPro1595) is added to each well of the protein A/G-coated plate and incubated for 1 hour at room temperature, allowing the sulfo-tagged biologic to bind to captured ADAs. After incubation, the plate is washed three times to remove unbound sulfo-tagged reagent, ensuring that only ADA-bound labels contribute to the signal. This detection step leverages the specificity of the protein A/G capture to minimize background noise. The plate is then read using an electrochemiluminescence instrument, preferably the MSD QuickPlex SQ 120, as provided in the kit's instructions. The instrument measures the signal intensity, which correlates with ADA concentration, completing the assay process.

For result interpretation, the screening assay signal is compared to a screening cut point factor, e.g., 1.06, established during validation, to determine if ADAs are present. A signal exceeding this threshold indicates a positive screening result. For the confirmatory assay, the percentage reduction in signal due to pre-incubation with the confirmatory reagent is calculated and compared to a confirmatory cut point of 10.2%. If the signal reduction exceeds 10.2%, the ADAs are confirmed as specific to the trimeric biologic. These cut points, derived from validation studies, ensure accurate classification of ADA presence and specificity. The kit's instructions provide detailed guidance on cut point application, ensuring consistent result interpretation across users.

This step-by-step procedure, supported by the kit's pre-configured components, enables efficient execution of both screening and confirmatory assays. The confirmatory assay's inclusion allows for regulatory-compliant specificity testing, while the screening assay provides a robust initial assessment of ADA presence. By following this procedure, users can achieve precise and reproducible ADA detection, leveraging the ACE-AG assay's innovative design to address the challenges of immunogenicity testing for self-assembling trimeric biologics.

### Advantages Over Prior Art

The Affinity Capture Elution-Protein A/G (ACE-AG) assay, as embodied in the present embodiments, represents a significant advancement over conventional bridging and standard Affinity Capture Elution (ACE) assays for detecting anti-drug antibodies (ADAs) against self-assembling trimeric biologics, such as XPro1595. Additionally, unlike direct ELISA or surface plasmon resonance-based methods, the ACE-AG assay's two-plate design and protein A/G capture provide superior specificity for multi-meric biologics by eliminating interference from free drug or reagent complexes. The primary limitation of bridging assays lies in their susceptibility to non-specific reagent interactions, particularly for trimeric biologics with dynamic monomer exchange. In bridging assays, biotinylated and sulfo-tagged drug reagents, intended to capture and detect ADAs, bind to each other in the absence of ADAs due to the biologic's self-assembling nature, resulting in high background signals and false positives. Standard ACE assays mitigate some non-specificity by eluting ADAs for detection on a separate platform, but they often fail to fully eliminate interference from reagent complexes, especially for biologics lacking an Fc region. The ACE-AG assay overcomes these challenges through a two-plate design that physically separates the capture and detection reagents, coupled with the use of protein A/G for specific immunoglobulin capture, ensuring minimal non-specific interactions and superior assay performance.

The ACE-AG assay's two-plate system is a cornerstone of its ability to eliminate non-specific reagent interactions. In the first plate, a streptavidin-coated surface pre-coated with biotinylated trimeric biologic captures ADAs from the sample, while the second plate, coated with protein A/G, captures only immunoglobulins from the eluted sample for detection with a sulfo-tagged trimeric biologic. This separation ensures that the biotinylated and sulfo-tagged reagents never interact directly, unlike in bridging assays where they form complexes in solution. Protein A/G's high affinity for IgGs and IgMs further enhances specificity by selectively binding ADAs, excluding free biologic or other non-specific components that might persist in standard ACE assays. This design enables the ACE-AG assay to deliver clear, specific signals, making it uniquely suited for trimeric biologics like XPro1595.

The ACE-AG assay's performance metrics underscore its superiority over bridging and standard ACE assays. It achieves a sensitivity of less than 2 ng/ml in screening assays and 1.752 ng/ml in confirmatory assays, allowing detection of low-level ADAs that might be obscured by background noise in bridging assays. The assay also demonstrates robust drug tolerance, detecting positive controls at 300, 100, and 8 ng/mL in the presence of up to 200 ng/mL of free trimeric biologic, a capability often limited in standard ACE assays due to residual reagent interference. Additionally, the assay exhibits no hook effect at ADA concentrations up to 65,536 ng/mL, ensuring accurate detection across a wide range of antibody levels, unlike bridging assays where high concentrations can reduce signal accuracy. These metrics, validated through rigorous testing, highlight the assay's ability to perform reliably under conditions that challenge conventional methods.

The robustness of the ACE-AG assay is further evidenced by its selectivity and performance in complex matrices. Validation studies showed no false positives in 10 random human serum lots, with all spiked samples (8 ng/mL surrogate antibody) testing positive, confirming high specificity. The assay's ability to function effectively in hemolyzed and lipemic serum demonstrates its resilience in challenging sample conditions, where bridging and standard ACE assays may falter due to matrix interference. Stability studies established that samples remain viable for ADA detection after 21.2 hours at benchtop conditions and 8 freeze/thaw cycles, enhancing the assay's practicality compared to less stable conventional methods.

In summary, the ACE-AG assay's innovative two-plate design and protein A/G capture mechanism eliminate the non-specific reagent interactions that plague bridging and standard ACE assays, offering a tailored solution for self-assembling trimeric biologics. Its superior sensitivity, drug tolerance, and robustness, position it as a significant improvement over conventional assays, capable of delivering precise and reliable ADA detection in diverse and challenging conditions.

### ALTERNATIVE EMBODIMENTS

While the preferred embodiment of the present invention employs a biotinylated form of the multi-meric biologic for capture and a sulfo-tagged form for detection within a two-plate system utilizing protein A/G, several alternative embodiments can be envisioned. These variations may be adapted to different experimental conditions or biologic properties, though they may not consistently achieve the specificity, sensitivity, or drug tolerance of the claimed method.

### Alternative Capture Mechanisms

In lieu of a biotinylated biologic for capturing ADAs, the multi-meric biologic may be immobilized on the first solid support via covalent bonding. For example, N-hydroxysuccinimide (NHS) esters can react with amine groups on the biologic, or maleimide can target thiol groups, providing a direct attachment that bypasses the need for biotin-streptavidin interactions. Alternatively, other affinity tags could be utilized, such as histidine tags (His-tags) paired with nickel- or cobalt-coated supports, or glutathione S-transferase (GST) tags with glutathione-coated supports, offering specific binding without relying on biotin. Another possibility involves direct adsorption of the biologic to the solid support through hydrophobic or electrostatic interactions; however, this approach may compromise specificity due to increased non-specific binding.

### Alternative Detection Strategies

For detecting captured ADAs, alternatives to the sulfo-tagged biologic can be employed. The biologic could be conjugated with fluorescent labels, such as fluorescein isothiocyanate (FITC) or Cy5, with the resulting fluorescence signal measured using a fluorometer or plate reader. Enzymatic labels, such as horseradish peroxidase (HRP) or alkaline phosphatase (AP), represent another option, where detection is achieved using colorimetric substrates (e.g., TMB for HRP) or chemiluminescent substrates (e.g., luminol for HRP or dioxetane for AP). Though less common, radioactive labels like iodine-125 or tritium could also be incorporated, with detection performed via scintillation counting or autoradiography.

### Variations in Plate Configuration

The two-plate system of the preferred embodiment can be altered in several ways. One variation involves conducting the assay on a single solid support, where the multi-meric biologic captures ADAs and detection occurs directly on the same plate. This simplification, however, may heighten the risk of non-specific binding, particularly with self-assembling biologics. Alternatively, the second solid support could be coated with immunoglobulin-binding proteins other than protein A/G, such as protein L (which binds kappa light chains) or species-specific anti-IgG antibodies, potentially altering the binding characteristics of the assay.

Direct Detection without Elution

In some embodiments, the elution step could be omitted entirely, with detection performed directly on the first solid support by adding the labeled detection reagent after washing away unbound sample components. While this eliminates a processing step, it may increase background signals due to residual non-specific interactions between the capture and detection reagents.

### CONSIDERATIONS AND LIMITATIONS

These alternative embodiments offer flexibility in assay design but may not match the performance of the preferred method, particularly for self-assembling multi-meric biologics where minimizing non-specific reagent interactions is paramount. The use of covalent attachment, alternative tags, or single-plate systems, for instance, could introduce challenges in maintaining the assay's sensitivity and drug tolerance, underscoring the advantages of the biotinylated and sulfo-tagged two-plate approach described in the claims.

### REFERENCES

### Patent Literature

U.S. Pat. No. 7,056,695 (Xencor, Inc.)
U.S. Pat. No. 7,101,974 (Xencor, Inc.)
U.S. Pat. No. 7,144,987 (Xencor, Inc.)
U.S. Pat. No. 7,244,823 (Xencor, Inc.)
U.S. Pat. No. 7,446,174 (Xencor, Inc.)
U.S. Pat. No. 7,662,367 (Xencor, Inc.)
U.S. Pat. No. 7,687,461 (Xencor, Inc.)
U.S. Pat. No. 11,365,299 (Xencor, Inc.)

### Non-Patent Literature

Steed, P. M., Tansey, M. G., Zalevsky, J., Zhukovsky, E. A., Desjarlais, J. R., Szymkowski, D. E., . . . & Latchman, Y. (2003). Inactivation of TNF signaling by rationally designed dominant-negative TNF variants. Science, 301 (5641), 1895-1898.
Zalevsky, J., Secher, T., Ezhevsky, S. A., Janot, L., Steed, P. M., O'Brien, C., . . . & Szymkowski, D. E. (2007). Dominant-negative inhibitors of soluble TNF attenuate experimental arthritis without suppressing innate immunity to infection. The Journal of Immunology, 179 (3), 1872-1883.
Johnson, D., et al. (2021). Sensitive assay design for detection of anti-drug antibodies to biotherapeutics that lack an immunoglobulin Fc domain. Scientific Reports, 11, 15467.
Bivi, N., et al. (2020). Development and validation of a novel immunogenicity assay to detect anti-drug and anti-PEG antibodies simultaneously with high sensitivity. Journal of Immunological Methods, 486, 112856.

What is claimed is:

1. A method for detecting anti-drug antibodies (ADAs) against a self-assembling multi-meric biologic in a biological sample, the method comprising:
a) contacting the biological sample with a biotinylated form of the multi-meric biologic coated on a first solid support to capture ADAs;
b) eluting the captured ADAs from the first solid support using an acidic solution;
c) transferring the eluted ADAs to a second solid support coated with protein A/G, wherein the protein A/G captures the eluted ADAs;
d) detecting the captured ADAs on the second solid support using a sulfo-tagged form of the multi-meric biologic; and
e) measuring a signal generated by the sulfo-tagged multi-meric biologic to determine the presence of ADAs,
wherein the multi-meric biologic comprises two or more monomeric subunits capable of reversible dissociation and association, and the method reduces non-specific binding between the biotinylated and sulfo-tagged forms of the multi-meric biologic to a level comparable to negative control samples, as compared to standard bridging assays.

2. The method of claim 1, wherein the multi-meric biologic is a pegylated, engineered variant of soluble human Tumor Necrosis Factor (TNF).

3. The method of claim 2, wherein the multi-meric biologic is XPro1595 (pegipanermin).

4. The method of claim 1, wherein the biological sample is human serum.

5. The method of claim 1, wherein the first solid support is a streptavidin-coated plate.

6. The method of claim 1, wherein the acidic solution comprises acetic acid at a concentration of 250-350 mM or an equivalent acidic solution capable of eluting ADAs without denaturation.

7. The method of claim 1, wherein the eluted ADAs are neutralized with a basic solution comprising 0.35M Tris at pH 9.5 prior to capture on the second solid support.

8. The method of claim 1, wherein the second solid support is a standard bind plate coated with protein A/G and blocked to reduce non-specific binding.

9. The method of claim 1, wherein the signal is measured using electrochemiluminescence.

10. The method of claim 1, wherein the biological sample is diluted at a ratio of 1:40 in an assay buffer prior to contacting the first solid support.

11. The method of claim 1, further comprising a confirmatory assay wherein the biological sample is contacted with the multi-meric biologic prior to step (a) to assess specificity of the ADAs.

12. The method of claim 1, wherein the method is selective for ADAs in hemolyzed or lipemic human serum samples.

13. The method of claim 1, wherein the biological sample is human plasma or cerebrospinal fluid, and the sample is diluted in assay buffer at a ratio of 1:10 to 1:40 prior to contacting the first solid support.

14. The method of claim 1, wherein the signal is measured using fluorescence or chemiluminescence, and the sulfo-tagged form of the multi-meric biologic is substituted with a fluorescein- or horseradish peroxidase-labeled form of the multi-meric biologic.

15. A kit for detecting anti-drug antibodies (ADAs) against a self-assembling multi-meric biologic in a biological sample, the kit comprising: a) a first solid support coated with a biotinylated form of the trimeric biologic; b) a second solid support coated with protein A/G; c) a sulfo-tagged form of the trimeric biologic for detection; d) an acidic elution solution; e) a neutralization solution; and f) instructions for performing the method of claim 1.

16. A method for detecting anti-drug antibodies (ADAs) against a self-assembling multi-meric biologic in a biological sample, the method comprising:
a) providing the multi-meric biologic immobilized on a first solid support via covalent attachment, an affinity tag, or adsorption;
b) contacting the biological sample with the immobilized biologic to capture ADAs;
c) either:
i) eluting the captured ADAs from the first solid support and transferring them to a second solid support coated with an immunoglobulin-binding protein, or
ii) retaining the captured ADAs on the first solid support;
d) detecting the ADAs using a labeled form of the multi-meric biologic, wherein the label is a fluorescent, enzymatic, or radioactive label.
